(19) United States
(12) Patent Application Publication
(10) Pub. No.: US 2021/0330556 A1
(43) Pub. Date: Oct. 28, 2021

(54) COSMETIC COMPOSITION INCLUDING MICROSPHERES CONTAINING HIGH CONCENTRATION OF OXYGEN AND METHOD OF PREPARING THE SAME

(71) Applicant: COIZ Co.,Ltd, Seoul (KR)

(72) Inventors: Jae-Hyun WOO, Seoul (KR); Hee-Nam CHAE, Jincheon-gun (KR)

(73) Assignee: COIZ Co.,Ltd, Seoul (KR)

(21) Appl. No.: 17/367,577

(22) Filed: Jul. 5, 2021

Related U.S. Application Data
(62) Division of application No. 16/914,460, filed on Jun. 28, 2020.

(30) Foreign Application Priority Data
Jul. 16, 2019 (KR) 10-2019-0085783

(51) Int. Cl. A61K 8/02; A61K 8/11; A61K 8/22; A61K 8/31; A61K 8/34; A61K 8/41; A61K 8/81; A61K 8/86; A61K 8/90; A61Q 19/08

(52) U.S. Cl. CPC A61K 8/025; A61K 8/11; A61K 8/22; A61K 8/315; A61K 8/345; A61K 8/41; A61K 2800/59; A61K 8/86; A61K 8/90; A61Q 19/08; A61K 2800/412; A61K 2800/524; A61K 8/8152

(57) ABSTRACT

The present invention provides a cosmetic composition including oxygen-containing microspheres for preventing skin aging, in which the microsphere contains 25 to 35 ppm of oxygen. The cosmetic composition including the microsphere according to the present invention is capable of supplying a high concentration of oxygen to the skin by allowing a high concentration of oxygen to slowly release through the microspheres.

### CROSS-REFERENCE TO RELATED APPLICATIONS

This application is a divisional application of U.S. patent application Ser. No. 16/914,460, entitled “COSMETIC COMPOSITION INCLUDING MICROSPHERES CONTAINING HIGH CONCENTRATION OF OXYGEN AND METHOD OF PREPARING,” filed on Jun. 28, 2020, which claims priority to and the benefit of Korean Patent Application No. 10-2019-0085783 filed in the Korean Intellectual Property Office on Jul. 16, 2019, the entire contents of which are incorporated herein by reference.

### TECHNICAL FIELD

The present invention relates to a cosmetic composition including microspheres, and more particularly, to a cosmetic composition including microspheres containing a high concentration of oxygen.

### BACKGROUND ART

Recently, interest in functional cosmetic materials containing specific bioactive substances for the purpose of suppressing skin aging and the like has increased and many studies have been conducted.

Particularly, the skin of the human body is a part directly exposed to the external environment, and is greatly affected by the external environment. Oxygen is a representative atmospheric component that affects the skin.

When oxygen is in an excessive state, oxygen is changed to the form of active oxygen to cause stress on the skin. The active oxygen means oxygen that is in an unstable state completely different from oxygen we breathe, and may be produced when oxygen is excessively produced due to environmental pollution, chemicals, ultraviolet rays, blood circulation disorders, stress, and the like. When the skin is excessively affected by active oxygen and the like, skin irritation and inflammatory reactions, such as erythema, edema, rash, tingling, and itching.

In the meantime, the skin is exposed to a hypoxic state when a person climbs a high mountain area or burn a stove in an enclosed space in winter, and in this case, oxygen may be insufficient to affect the skin. In case of severe oxygen deficiency, oxygen and nutrient are insufficiently supplied to hair follicle cells to cause alopecia and cause abnormal proliferation of tissue known as keloid skin. When the hypoxic situation continues, it also affects collagen synthesis to reduce skin elasticity.

According to a study published in the international journal “Spine” in 2011, as a result of placing human skin synthetic cells in “0% oxygen” and “21% oxygen” environments for three days, about 25% less collagen molecules are produced in an oxygen-free environment, and the structural characteristics of the produced collagen molecules are also worse. In this study, it was confirmed that the amount of oxygen plays an important role in collagen formation, and specifically, it was found that substances, such as “H1F 1α”, produced by cells when oxygen was insufficient inhibited collagen synthesis. Further, when there is a constant problem with the supply of oxygen to the cells, the skin may be necrotic, a typical example of which is “diabetes feet”, and due to diabetes complications, peripheral blood vessels are broken and blood circulation is poor, and when the amount of oxygen supply is reduced, tissues die and foot ulcers or infections do not heal well.

Conventionally, most of the skin diseases caused by oxygen deficiency have been treated with surgery or drugs, but recently, a method of directly using oxygen has been proposed. The problem caused due to oxygen is solved by “oxygen supply”. Oxygen corresponding to a high concentration in an appropriate amount acts a metabolic substrate and signal molecule to play an important role in maintaining in vivo homeostasis and healing wounds. Hyperbaric oxygen temporarily increases a partial pressure of oxygen in the cell and increases active oxygen to promote cell proliferation. Hyperbaric oxygen promotes collagen synthesis and blood vessel production by lysyl oxidase based on oxygen, and makes growth factors that promote angiogenesis be secreted or allows stem cells to migrate from the bone marrow to promote angiogenesis or wound healing. Directly supplying oxygen to the skin may be an effective method for delaying aging on the one hand. Research conducted in this regard has suggested a method of treating skin wounds by dissolving hyperbaric oxygen in water. In 2013, a research tem at the University of New Brunswick's School of Physical Epidemiology in Canada immersed the feel of a diabetic patient in hyperbaric oxygen-dissolved water for 30 minutes, and as a result, it showed that partial pressure of oxygen in the skin increases three times or more from 65 mmHg to 205 mmHg, and it was confirmed that the method of dissolving oxygen in water and directly supplying oxygen-dissolved water to the skin also has the effect of supplying oxygen to the skin.

In general, a concentration of oxygen in water is about 4 to 8 ppm, so that a physical or chemical treatment is required to ensure the concentration of oxygen is 10 to 15 times or higher in water. Similar to the principle of producing carbonated water, oxygen water containing hyperbaric oxygen may be produced by a pressurization method. However, in the case of the foregoing method, there is a problem in that when pressure is removed, the concentration of oxygen is rapidly decreased. Further, as a material for producing oxygen for locally transferring oxygen, hydrogen peroxide, sodium percarbonate, calcium peroxide, and the like may also be used, but these materials also have a rapid oxygen releases at an initial stage, so that it is difficult to expect the effect of continuously supplying oxygen.

### SUMMARY OF THE INVENTION

The present invention has been made in an effort to provide a cosmetic composition including microspheres containing a high concentration of oxygen for supplying a high concentration of oxygen to the skin by allowing the high concentration of oxygen to gradually release through the microspheres containing oxygen.

The objects of the present invention are not limited to the foregoing objects, and those skilled in the art will clearly understand other non-mentioned objects through the description below.

An aspect of the present invention provides a cosmetic composition including oxygen-containing microspheres for preventing skin aging, in which the microsphere contains 25 to 35 ppm of oxygen.

The microsphere may be produced by: homogenizing about 5.0 to 6.0 parts by weight of poloxamer 188 and 1.5 to 2.5 parts by weight of glycerin with respect to 100 parts by weight of purified water under a condition of about 80 to 90° C.; cooling the homogenized aqueous solution to 23 to 27° C.; adding 25 to 55 parts by weight of oxygen water in which 95 to 105 ppm of oxygen is dissolved to the cooled aqueous solution and stirring the solution; and mixing the stirred solution with 35 to 69 parts by weight of a fluorine compound selected from the group consisting of perfluorodecalin, methyl perfluoroisobutyl ether, and perfluoropolymethylisopropyl ether under a condition of 20 to 30° C., and making the mixture pass through a high pressure homogenizer.

The microsphere may be produced by adding one or more materials selected from the group consisting of acrylate/C10-30alkyl acrylate cross polymer, polysorbate, triethanolamine, a flavoring agent, and a preservative to the microsphere in an appropriate amount, and dispersing and stabilizing the material, following by aging for 2 to 7 days.

The perfluorodecalin may be 25 to 55 parts by weight, and the perfluoropolymethylisopropyl ether may be 10 to 14 parts by weight.

The present invention may provide a cosmetic composition including microspheres containing a high concentration of oxygen for supplying a high concentration of oxygen to the skin by allowing the high concentration of oxygen to gradually release through the microspheres containing oxygen.

The effects of the present invention are not limited to the foregoing effects, and those skilled in the art will clearly understand other non-mentioned effects through the description below.

### DETAILED DESCRIPTION

Objects, effects, and technical configurations for achieving them will be clear when exemplary embodiments described in detail with reference to the accompanying drawings are referred to. In the following description of the present invention, a detailed description of known functions and configurations incorporated herein is omitted to avoid making the subject matter of the present invention unclear. In addition, the terminology used in the description is defined in consideration of the function of corresponding components used in the present invention and may be varied according to users.

However, the present invention is not limited to exemplary embodiments disclosed herein but will be implemented in various forms. The exemplary embodiments are provided so that the present invention is completely disclosed, and a person of ordinary skilled in the art can fully understand the scope of the present invention, and the present invention will be defined only by the scope of the appended claims. Accordingly, the definitions thereof should be made based on the entire contents of the present specification.

Hereinafter, a cosmetic composition including microspheres according to an exemplary embodiment of the present invention will be described in detail with reference to the accompanying drawings.

A cosmetic composition including microspheres according to an exemplary embodiment of the present invention includes about 25 to 35 ppm oxygen. The microsphere has excellent skin penetration and may supply oxygen so that oxygen can be stably preserved without losing long-term activity.

The microsphere may be produced by encapsulating oxygen water containing about 100 ppm or more of oxygen water and adding the encapsulated microsphere to a cosmetic material.

More particularly, the microsphere may be produced by homogenizing about 5.0 to 6.0 parts by weight of poloxamer 188 and about 1.5 to 2.5 parts by weight of glycerin with respect to 100 parts by weight of purified water under a condition of about 80 to 90° C., cooling the homogenized aqueous solution to about 23 to 27° C., and adding about 25 to 55 parts by weight of oxygen water in which about 95 to 105 ppm of oxygen is dissolved to the cooled aqueous solution and stirring the solution. Next, the stirred solution is mixed with about 35 to 69 parts by weight of a fluorine compound selected from the group consisting of perfluorodecalin (PFC), methyl perfluoroisobutyl ether, and perfluoropolymethylisopropyl ether under a condition of about 20 to 30° C., and the mixture passes through a high pressure homogenizer. Methyl perfluoroisobutyl ether is the component usable while being be replaced with perfluorodecarine, and may be replaced with perfluorodecarine in the same amount as that of the methyl perfluoroisobutyl ether for use.

Herein, perfluorodecaline (C₁₀F₁₈) is a compound of fluorine and carbon and is a material that dissolves the large amount of oxygen. Perfluorodecaline has been known as a material capable of dissolving about 21% (up to 45%) of oxygen that is similar to the concentration of oxygen in the atmosphere.

Perfluorodecaline is a component for containing a high concentration of oxygen for producing the microsphere, and may supply a high concentration of oxygen to the microsphere by making perfluorodecaline and perfluoropolymethylisopropyl ether pass through the high pressure homogenizer together or separately.

An emulsification stabilization supplement may be contained for stabilizing the oxygen containing microsphere. Examples of the emulsification stabilization supplement may include a polymer, such as glycerin, xylitol, and a natural polymer compound. In this case, the emulsification stabilization supplement may be used in the amount of about 0.1 to 3.0 parts by weight with respect to the total weight of the composition.

A concentration of oxygen contained in the microsphere may be about 30 ppm, and when the concentration of oxygen is equal to or less than 30 ppm, the microsphere is processed until the concentration of oxygen becomes about 30 ppm by additionally adding about 5 to 20 parts by weight of the oxygen water (100 ppm) and making the solution pass through the high pressure homogenizer.

By the cosmetic composition including the microsphere according to the exemplary embodiment of the present invention, it is possible to mix oxygen to a cosmetic material in a higher concentration state than before, and improve permeability when the cosmetic composition is applied to the skin, thereby providing the cosmetic material effective to the skin.

The present invention will be described in more detail through the following Examples, but the present invention is not limited by the following Examples.

<Method of Producing Microsphere>

### Example 1

A. Prepare Stirred Solution

With respect to 100 parts by weight of purified water under a condition of about 80 to 90° C., about 5.5 parts by weight of poloxamer 188 and about 2 parts by weight of glycerin were homogenized, followed by cooling the homogenized aqueous solution to about 23 to 27° C.

Oxygen water in which about 100 ppm of oxygen was dissolved was added to the cooled aqueous solution by about 30 parts by weight and stirred to prepare a stirred solution.

B. Produce Microsphere

Next, the stirred solution, about 50 parts by weight of perfluorodecalin, and about 12 parts by weight of perfluoropolymethylisopropyl ether were mixed under a condition of about 20 to 30° C. at 900 to 1,100 rpm, followed by making the mixture pass through a high pressure homogenizer.

### Example 2

A. Prepare Stirred Solution

With respect to 100 parts by weight of purified water under a condition of about 80 to 90° C., about 5.5 parts by weight of poloxamer 188 and about 2 parts by weight of glycerin were homogenized, followed by cooling the homogenized aqueous solution to about 23 to 27° C.

Oxygen water in which about 100 ppm of oxygen was dissolved was added to the cooled aqueous solution by about 30 parts by weight and stirred to prepare a stirred solution.

B. Produce Microsphere

Next, the stirred solution, about 30 parts by weight of perfluorodecalin, and about 12 parts by weight of perfluoropolymethylisopropyl ether were mixed under a condition of about 20 to 30° C. at 900 to 1,100 rpm, followed by making the mixture pass through a high pressure homogenizer.

### Example 3

A. Prepare Stirred Solution

With respect to 100 parts by weight of purified water under a condition of about 80 to 90° C., about 5.5 parts by weight of poloxamer 188 and about 2 parts by weight of glycerin were homogenized, followed by cooling the homogenized aqueous solution to about 23 to 27° C.

Oxygen water in which about 100 ppm of oxygen was dissolved was added to the cooled aqueous solution by about 30 parts by weight and stirred to prepare a stirred solution.

B. Produce Microsphere

Next, the stirred solution, about 15 parts by weight of perfluorodecalin, and about 6 parts by weight of perfluoropolymethylisopropyl ether were mixed under a condition of about 20 to 30° C. at 900 to 1,100 rpm, followed by making the mixture pass through a high pressure homogenizer.

### Example 4

A. Prepare Stirred Solution

With respect to 100 parts by weight of purified water under a condition of about 80 to 90° C., about 5.5 parts by weight of poloxamer 188 and about 2 parts by weight of glycerin were homogenized, followed by cooling the homogenized aqueous solution to about 23 to 27° C.

Oxygen water in which about 100 ppm of oxygen was dissolved was added to the cooled aqueous solution by about 30 parts by weight and stirred to prepare a stirred solution.

B. Produce Microsphere

Next, the stirred solution and about 32 parts by weight of perfluoropolymethylisopropyl ether were mixed under a condition of about 20 to 30° C. at 900 to 1,100 rpm, followed by making the mixture pass through a high pressure homogenizer.

### Comparative Example 1

A. Prepare Stirred Solution

With respect to 100 parts by weight of purified water under a condition of about 80 to 90° C., about 0.2 parts by weight of acrylate/C10-30 alkyl acrylate cross polymer, about 0.2 parts by weight of polysorbate, and about 0.2 parts by weight of triethanolamine were homogenized, followed by cooling the homogenized aqueous solution to about 23 to 27° C.

Oxygen water in which about 100 ppm of oxygen was dissolved was added to the cooled aqueous solution by about 30 parts by weight and stirred to prepare a stirred solution.

B. Produce Microsphere

Next, the stirred solution and about 50 parts by weight of perfluorodecalin were mixed under a condition of about 20 to 30° C. at 900 to 1,100 rpm, followed by making the mixture pass through a high pressure homogenizer.

### Comparative Example 2

A. Prepare Stirred Solution

With respect to 100 parts by weight of purified water under a condition of about 80 to 90° C., about 0.2 parts by weight of acrylate/C10-30 alkyl acrylate cross polymer, about 0.2 parts by weight of polysorbate, and about 0.2 parts by weight of triethanolamine were homogenized, followed by cooling the homogenized aqueous solution to about 23 to 27° C.

Oxygen water in which about 100 ppm of oxygen was dissolved was added to the cooled aqueous solution by about 30 parts by weight and stirred to prepare a stirred solution.

B. Produce Microsphere

Next, the stirred solution and about 30 parts by weight of perfluorodecalin were mixed under a condition of about 20 to 30° C. at 900 to 1,100 rpm, followed by making the mixture pass through a high pressure homogenizer.

### Comparative Example 3

A. Prepare Stirred Solution

With respect to 100 parts by weight of purified water under a condition of about 80 to 90° C., about 0.2 parts by weight of acrylate/C10-30 alkyl acrylate cross polymer, about 0.2 parts by weight of polysorbate, and about 0.2 parts by weight of triethanolamine were homogenized, followed by cooling the homogenized aqueous solution to about 23 to 27° C.

Oxygen water in which about 100 ppm of oxygen was dissolved was added to the cooled aqueous solution by about 30 parts by weight and stirred to prepare a stirred solution.

B. Produce Microsphere

Next, the stirred solution and about 15 parts by weight of perfluorodecalin were mixed under a condition of about 20 to 30° C. at 900 to 1,100 rpm, followed by making the mixture pass through a high pressure homogenizer.

### Comparative Example 4

A. Prepare Stirred Solution

With respect to 100 parts by weight of purified water under a condition of about 80 to 90° C., about 0.2 parts by weight of acrylate/C10-30 alkyl acrylate cross polymer, about 0.2 parts by weight of polysorbate, and about 0.2 parts by weight of triethanolamine were homogenized, followed by cooling the homogenized aqueous solution to about 23 to 27° C.

Oxygen water in which about 100 ppm of oxygen was dissolved was added to the cooled aqueous solution by about 30 parts by weight and stirred to prepare a stirred solution.

B. Produce Microsphere

Next, the stirred solution and about 32 parts by weight of perfluoropolymethylisopropyl ether were mixed under a condition of about 20 to 30° C. at 900 to 1,100 rpm, followed by making the mixture pass through a high pressure homogenizer.

Table 1 below represents a composition ratio according to the production of the microsphere.

TABLE 1

| | | Example | | | | Comparative Example | | | |
|---|---|---|---|---|---|---|---|---|---|
| NO. | Component | 1 | 2 | 3 | 4 | 1 | 2 | 3 | 4 |
| 1 | Purified water | 100 | 100 | 100 | 100 | 100 | 100 | 100 | 100 |
| 2 | Oxgyen water (100 | 30 | 30 | 30 | 30 | 30 | 30 | 30 | 30 |
| | ppm) | | | | | | | | |
| 3 | Poloxamer 188 | 5.4 | 5.4 | 5.4 | 5.4 | — | — | — | — |
| 4 | Glycerin | 2 | 2 | 2 | 2 | — | — | — | — |
| 5 | Perfluorodecalin | 50 | 30 | 15 | — | 50 | 30 | 15 | — |
| 6 | Perfluoropolymethyl- | 12 | 12 | 6 | 32 | — | — | — | 32 |
| | isopropyl ether | | | | | | | | |
| 7 | Acrylate/C10-30 alkyl | — | — | — | — | 0.2 | 0.2 | 0.2 | 0.2 |
| | acrylate cross polymer | | | | | | | | |
| 8 | Polysorbate | — | — | — | — | 0.2 | 0.2 | 0.2 | 0.2 |
| 9 | Triethanolamine | — | — | — | — | 0.2 | 0.2 | 0.2 | 0.2 |

Table 2 below represents a measurement result of oxygen concentrations of the microspheres according to Examples 1 to 4 and Comparative Examples 1 to 4.

TABLE 2

| Experimental | Example | Example | Example | Example | Comparative | Comparative | Comparative | Comparative |
|---|---|---|---|---|---|---|---|---|
| Example | 1 | 2 | 3 | 4 | Example 1 | Example 2 | Example 3 | Example 4 |
| Oxygen | 35 | 37 | 33 | 31 | 13 | 14 | 10 | 10 |
| concentration | | | | | | | | |
| (ppm) | | | | | | | | |

<Prepare Cosmetic Composition>

### Examples 5-1 to 5-4

With respect to 100 parts by weight of purified water under a condition of about 20 to 30° C., about 3.0 parts by weight of polyacrylamide/C12 isoparaffin/laures-7 was mixed, and about 20.0 parts by weight of almond oil was added and mixed slowly.

The mixed solution and about 10 parts by weight of the microspheres produced according to Examples 1 to 4 were mixed and aged for about 2 to 7 days.

### Comparative Example 5

With respect to 100 parts by weight of purified water under a condition of about 20 to 30° C., about 3.0 parts by weight of polyacrylamide/C12 isoparaffin/laures-7 was mixed, and about 20.0 parts by weight of almond oil was added and mixed slowly.

The mixed solution and about 5.0 parts by weight of perfluorodecalin were mixed and aged for about 2 to 7 days.

Table 3 below represents the composition ratios of Example 5 and Comparative Example 5.

TABLE 3

| | | Examples 5-1 | Comparative |
|---|---|---|---|
| NO. | Component | to 5-4 | Example 5 |
| 1 | Purified water | 100 | 100 |
| 2 | Perfluorodecalin | — | 5.0 |
| 3 | Microspheres of Examples 1 to 4 | 10.0 | — |
| 4 | Almond oil | 20.0 | 20.0 |
| 5 | Polyacrylamide/C12 | 3.0 | 3.0 |
| | isoparaffin/laures-7 | | |

<Centrifugal Stability Test>

In order to examine the centrifugal stability of the cosmetic compositions prepared according to Examples 5-1 to 5-4 and Comparative Example 5, each product was prepared by about 500 g, and the appropriate and same amount of cosmetic composition was centrifuged at about 4,000 RPM for about 10 minutes, about 8,000 RPM for about 10 minutes, and about 12,000 RPM for about 10 minutes, and the results thereof are represented in Table 3.

Table 4 represents a result of the centrifugal stability test.

TABLE 4

| Experimental | 4,000 RPM | 8,000 RPM | 12,000 RPM |
|---|---|---|---|
| Example | (10 minutes) | (10 minutes) | (10 minutes) |
| Example 5-1 | Stable | Stable | Stable |
| Example 5-2 | Stable | Stable | Stable |
| Example 5-3 | Stable | Stable | Stable |
| Example 5-4 | Stable | Stable | Stable |
| Comparative | Small-amount | Separate | — |
| Example 5 | separate | | |

As can be seen in the result of the centrifugal stability test of the table, it was confirmed that Examples 5-1 to 5-4 for the cosmetic composition including the microsphere containing oxygen had no problem in centrifugal stability compared to Comparative Example 5 containing oxygen.

<Temporal Stability Test>

In order to examine temporal stability of the cosmetic compositions prepared according to Examples 5-1 to 5-4 and Comparative Example 5, about 500 g of each product was contained in a transparent glass container and stored at a room temperature (about 25° C.), a high temperature (about 45° C.), and sunlight and freezing-thawing room (45° C. to −10° C., 24 hour cycle), and the obtained results are represented in Table 5.

TABLE 5

| | | | | | | Comparative |
|---|---|---|---|---|---|---|
| Storage condition | | Example 5-1 | Example 5-2 | Example 5-3 | Example 5-4 | Example 5 |
| About | 1 month | Stable | Stable | Stable | Stable | Stable |
| 25° C. | 3 months | Stable | Stable | Stable | Stable | Separate |
| | 6 months | Stable | Stable | Stable | Stable | — |
| | 1 year | Stable | Stable | Stable | Stable | — |
| About | 1 month | Stable | Stable | Stable | Stable | Stable |
| 45° C. | 3 months | Stable | Stable | Stable | Stable | Separate |
| | 6 months | Stable | Stable | Stable | Stable | — |
| | 1 year | Stable | Stable | Stable | Stable | — |
| Sunlight | 1 month | Stable | Stable | Stable | Stable | Stable |
| | 3 months | Stable | Stable | Stable | Stable | Separate |
| | 6 months | Stable | Stable | Stable | Stable | — |
| | 1 year | Stable | Stable | Stable | Stable | — |
| About | 1 week | Stable | Stable | Stable | Stable | Stable |
| 45° C. <--> | 2 weeks | Stable | Stable | Stable | Stable | Stable |
| About | 3 weeks | Stable | Stable | Stable | Stable | Separate |
| −10° C. | 4 weeks | Stable | Stable | Stable | Stable | — |

<Nutritional Cream Formulation Including Microsphere>

### Formulation Examples 1-1 to 1-4

About 2.0 parts by weight of wax, about 1.5 parts by weight of polysorbate 60, about 0.8 parts by weight of sorbitan sesquioleate, about 5.0 parts by weight of liquid paraffin, about 5.0 parts by weight of squalene, about 4.0 parts by weight of caprylic/capric triglyceride, and about 0.2 parts by weight of triethanolamine were inserted while mixing and stirring about 0.2 parts by weight of carboxy vinyl polymer, about 5.0 parts by weight of glycerin, about 3.0 parts by weight of butylene glycol, about 3.0 parts by weight of propylene glycol, followed by emulsification by heating at about 80 to 85° C.

After the emulsification is completed, the mixture was stirred and cooled to about 40° C., and then the microspheres according to Examples 1 to 4 were inserted, and the mixture was cooled to about 25° C. while stirring the mixture with a paddle mixer at about 25 rpm and the aged.

### Comparative Formulation Example 1

About 2.0 parts by weight of wax, about 1.5 parts by weight of polysorbate 60, about 0.8 parts by weight of sorbitan sesquioleate, about 5.0 parts by weight of liquid paraffin, about 5.0 parts by weight of squalene, about 4.0 parts by weight of caprylic/capric triglyceride, and about 0.2 parts by weight of triethanolamine were inserted while mixing and stirring about 0.2 parts by weight of carboxy vinyl polymer, about 5.0 parts by weight of glycerin, about 3.0 parts by weight of butylene glycol, about 3.0 parts by weight of propylene glycol, followed by emulsification by heating at about 80 to 85° C.

After the emulsification is completed, the mixture was stirred and cooled to about 40° C., followed by aging.

Table 6 below represents the composition ratios of Formulation Examples 1-1 to 1-4 and Comparative Formulation Example 1.

TABLE 6

| | | | Comparative |
|---|---|---|---|
| | | Formulation | Formulation |
| NO. | Raw material | Example 1 | Example 1 |
| 1 | Microsphere (Examples 1-4) | 10.0 | — |
| 2 | Wax | 2.0 | 2.0 |
| 3 | Polysorbate 60 | 1.5 | 1.5 |
| 4 | Sorbitan sesquioleate | 0.8 | 0.8 |
| 5 | Liquid paraffin | 5.0 | 5.0 |
| 6 | Squalene | 5.0 | 5.0 |
| 7 | Caprylic/capric triglyceride | 4.0 | 4.0 |
| 8 | Carboxy vinyl polymer | 0.2 | 0.2 |
| 9 | Glycerin | 5.0 | 5.0 |
| 10 | Butylene glycol | 3.0 | 3.0 |
| 11 | Propylene glycol | 3.0 | 3.0 |
| 12 | Triethanolamine | 0.2 | 0.2 |

The following preference investigation was conducted to identify the effect of improving the skin condition when the nutritional creams of Formulation Examples 1-1 to 1-4 and Comparative Formulation Example 1-1 were applied to the skin. The people who participated in the test were female groups of about 20 to 55 years old, who consisted of 50 of normal, oily, dry, and complex skins, each composed of 25%, to examine the skin condition improvement and skin oil and moisture conditions. To the same person, the person applied the nutritional cream of Formulation Example 1 to the skin at the left side of the face and applied the nutritional cream of Comparative Formulation Example 1 to the skin at the right side of the face every morning for 20 days once a day after washing the face, and in order to identify the improvement of the skin condition, a survey was conducted on the degree of skin condition improvement. The test result is represented in Table 7 below.

TABLE 7

| | | Skin condition improvement | |
|---|---|---|---|
| Classification | | Number of people | % |
| Formulation | Very good | 31 | 62.0 |
| Example 1 | Good | 14 | 28.0 |
| | Normal | 5 | 10.0 |
| | So-so | 0 | 0.0 |
| Comparative | Very good | 5 | 10.0 |
| Formulation | Good | 18 | 36.0 |
| Example 1 | Normal | 16 | 32.0 |
| | So-so | 11 | 22.0 |

As represented in the survey result, it can be seen that Formulation Example 1 including the microsphere containing oxygen has the excellent skin improvement effect, compared to the case of Comparative Formulation Example 1.

The exemplary embodiments of the present invention are disclosed in the present specification and the drawings and the specific terms are used. However, the specific terms are used as general meanings simply for the purpose of clearly describing the present invention and helping the understanding of the invention, and do not intend to limit the scope of the present invention. It is obvious to those skilled in the art that other modification examples based on the technical spirit of the present invention, in addition to the exemplary embodiment disclosed herein may be carried out.

What is claimed is:

1. A method of producing a cosmetic composition including oxygen-containing microspheres, the method comprising:
producing oxygen-containing microspheres; and
adding an appropriate amount of one or more materials selected from selected from the group consisting of acrylate/C10-30alkyl acrylate cross polymer, polysorbate, triethanolamine, a flavoring agent, and a preservative to the oxygen-containing microspheres in an appropriate amount, and dispersing and stabilizing the material, following by aging for 2 to 7 days,
wherein the step of producing of the oxygen-containing microspheres includes:
homogenizing 5.0 to 6.0 parts by weight of poloxamer 188 and 1.5 to 2.5 parts by weight of glycerin with respect to 100 parts by weight of purified water under a temperature condition of 80 to 90° C.;
cooling the homogenized aqueous solution to 23 to 27° C.;
adding 25 to 55 parts by weight of oxygen water in which 95 to 105 ppm of oxygen is dissolved to the cooled aqueous solution and stirring the solution; and
mixing the stirred solution with 35 to 69 parts by weight of one or more fluorine compounds selected from the group consisting of perfluorodecalin, methyl perfluoroisobutyl ether, and perfluoropolymethylisopropyl ether under a temperature condition of 20 to 30° C., and making the mixture pass through a high pressure homogenizer.
